(19) United States
(12) Patent Application Publication
(10) Pub. No.: US 2002/0107250 A1
(43) Pub. Date: Aug. 8, 2002

(54) Rapid-onset formulation of a selective cyclooxygenase-2 inhibitor

(76) Inventors: Madhusudan Hariharan, Evanston, IL (US); Tugrul T. Kararli, Skokie, IL (US); Fred Hassan, Peapack, NJ (US); James C. Forbes, Glenview, IL (US)

Correspondence Address: Pharmacia Corporation, P.O. Box 5110, Chicago, IL, 60680-5110, US

(21) Appl. No.: 09/836,905

(22) Filed: Apr. 17, 2001

Related U.S. Application Data
(60) Provisional application No. 60/197,746, filed on Apr. 18, 2000.

(51) Int. Cl. A61K 31/50; A61K 31/415; A61K 31/353; A61K 31/122

(52) U.S. Cl. 514/247; 514/406; 514/456; 514/690

(57) ABSTRACT

An orally deliverable pharmaceutical composition is provided comprising a selective cyclooxygenase-2 inhibitory drug of low water solubility, for example celecoxib, and a glycol ether, for example diethylene glycol monoethyl ether. At least a substantial part of the drug is in dissolved or solubilized form in a solvent liquid comprising the glycol ether. The composition has rapid-onset properties and is useful in treatment of cyclooxygenase-2 mediated conditions and disorders, particularly pain. For relief of pain in headache or migraine, the composition can optionally be administered together with a vasodilator.

This application claims priority of U.S. provisional application Serial No. 60/197,746 filed on Apr. 18, 2000.

### FIELD OF THE INVENTION

The present invention relates to orally deliverable pharmaceutical compositions containing a selective cyclooxygenase-2 (COX-2) inhibitory drug, to processes for preparing such compositions, to methods of treatment comprising orally administering such compositions to a subject in need thereof, and to the use of such compositions in the manufacture of medicaments.

### BACKGROUND OF THE INVENTION

Numerous compounds have been reported having therapeutically and/or prophylactically useful selective COX-2 inhibitory effect, and have been disclosed as having utility in treatment or prevention of specific COX-2 mediated disorders or of such disorders in general. Among such compounds are a large number of substituted pyrazolyl benzenesulfonamides as reported in U.S. Pat. No. 5,466,823 to Talley et al., including for example the compound 4-[5-(4-methylphenyl)-3-(trifluoromethyl)-1H-pyrazol-1-yl]benzenesulfonamide, also referred to herein as celecoxib (I), and the compound 4-[5-(3-fluoro-4-methoxyphenyl)-3-difluoromethyl)-1H-pyrazol-1-yl]benzenesulfonamide, also referred to herein as deracoxib (II).

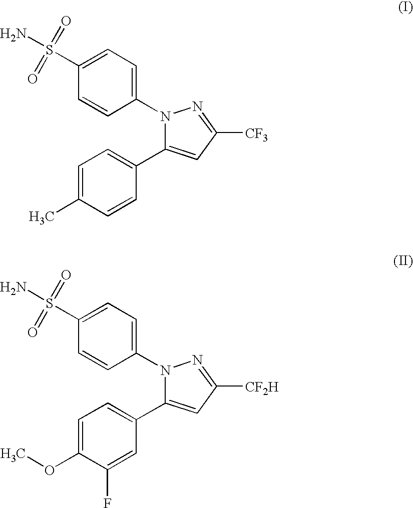

Other compounds reported to have therapeutically and/or prophylactically useful selective COX-2 inhibitory effect are substituted isoxazolyl benzenesulfonamides as reported in U.S. Pat. No. 5,633,272 to Talley et al., including the compound 4-[5-methyl-3-phenylisoxazol-4-yl]benzenesulfonamide, also referred to herein as valdecoxib (III).

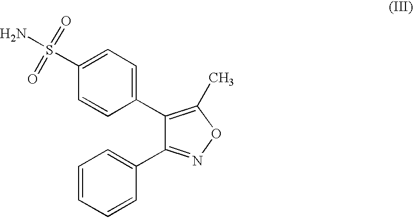

Still other compounds reported to have therapeutically and/or prophylactically useful selective COX-2 inhibitory effect are substituted (methylsulfonyl)phenyl furanones as reported in U.S. Pat. No. 5,474,995 to Ducharme et al., including the compound 3-phenyl-4-[4-(methylsulfonyl)phenyl]-5H-furan-2-one, also referred to herein as rofecoxib (IV).

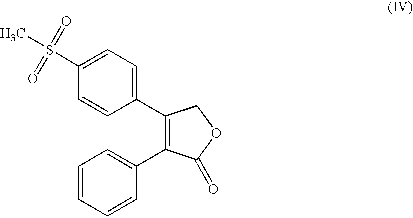

U.S. Pat. No. 5,981,576 to Belley et al. discloses a further series of (methylsulfonyl)phenyl furanones said to be useful as selective COX-2 inhibitory drugs, including 3-(1-cyclopropylmethoxy)-5,5-dimethyl-4-[4-(methylsulfonyl)phenyl]-5H-furan-2-one and 3-(1-cyclopropylethoxy)-5,5-dimethyl-4-[4-(methylsulfonyl)phenyl]-5H-furan-2-one.

U.S. Pat. No. 5,861,419 to Dube et al. discloses substituted pyridines said to be useful as selective COX-2 inhibitory drugs, including for example the compound 5-chloro-3-(4-methylsulfonyl)phenyl-2-(2-methyl-5-pyridinyl)pyridine, also referred to herein as etoricoxib (V).

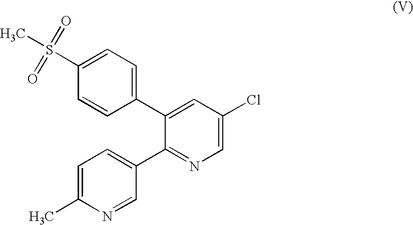

European Patent Application No. 0 863 134 discloses the compound 2-(3,5-difluorophenyl)-3-[4-(methylsulfonyl)phenyl]-2-cyclopenten-1-one said to be useful as a selective COX-2 inhibitory drug.

U.S. Pat. No. 6,034,256 to Carter et al. discloses a series of benzopyrans said to be useful as selective COX-2 inhibitory drugs, including the compound (S)-6,8-dichloro-2-(trifluoromethyl)-2H-1-benzopyran-3-carboxylic acid (VI).

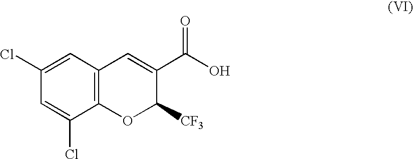

International Patent Publication No. WO 00/24719 discloses substituted pyridazinones said to be useful as selective COX-2 inhibitory drugs, including the compound 2-(3,4-difluorophenyl)-4-(3-hydroxy-3-methyl-1-butoxy)-5-[4-(methylsulfonyl)phenyl]-3-(2H)-pyridazinone.

Australian Patent Applications No. 200042711, No. 200043730 and No. 200043736 disclose compositions comprising a selective COX-2 inhibitory drug, a 5HT₁ receptor agonist and caffeine, said to be useful for treating migraine.

A need for formulated compositions of selective COX-2 inhibitory drugs, particularly rapid-onset compositions of such drugs, exists. Rapid-onset drug delivery systems can provide many benefits over conventional dosage forms. Generally, rapid-onset preparations provide a more immediate therapeutic effect than standard dosage forms. For example, in the treatment of acute pain, for example in headache or migraine, rapid-onset dosage forms would be useful to provide fast pain relief.

U.S. Pat. No. 5,993,858 to Crison & Amidon discloses an excipient formulation for increasing bioavailability of a poorly water-soluble drug. The formulation is said to be self-microemulsifying and to comprise an oil or other lipid material, a surfactant and a hydrophilic co-surfactant. The choice of surfactant is said to be less critical than the choice of co-surfactant, which reportedly should have an HLB (hydrophilic-lipophilic balance) number greater than 8. A preferred example of such a co-surfactant is said to be Labrasol™ of Gattefossé, identified as a product “comprised of medium-chain triglycerides derived from coconut oil” having HLB of 14. A formulation prepared containing 15 mg nifedipine in a size 1 (0.5 ml) capsule, i.e., at a concentration of 30 mg/ml, is described as a “clear solution” at 70° C. but a “semi-solid” at room temperature.

Cited in above-referenced U.S. Pat. No. 5,993,858 is prior work by Farah et al. in which a self-microemulsifying formulation was investigated for improving in vitro dissolution of indomethacin. The formulation of Farah et al. reportedly comprised an oil phase material Gelucire™ of Gattefossé, together with a polyethylene glycol capric/caprylic glyceride product having HLB of 10, a propylene glycol laurate product having HLB of 4, and diethylene glycol monoethyl ether.

U.S. Pat. No. 5,342,625 to Hauer et al. discloses microemulsion and microemulsifiable concentrate formulations of a cyclosporin. Such formulations are disclosed to comprise a glycol ether, for example diethylene glycol monoethyl ether.

Drugs of low water solubility are sometimes orally administered in suspension in an imbibable aqueous liquid. For example, a suspension of particulate celecoxib in a vehicle of apple juice is disclosed in co-assigned Ecuador Patent Application No. 98-2761, published on May 6, 1999 and incorporated herein by reference. Also disclosed in that application is a dilute solution of celecoxib in a mixture of PEG-400 (polyethylene glycol having an average molecular weight of about 400) and water in a 2:1 ratio by volume.

The suspension and solution compositions of Ecuador Patent Application No. 98-2761 are indicated therein to have comparable bioavailability. However, following oral administration to dogs, the time taken for blood serum celecoxib concentration to reach a maximum level (Tₘₐₓ) was shorter for the solution composition than for the suspension.

Above-cited U.S. Pat. No. 5,760,068 discloses that its subject pyrazolyl benzenesulfonamide compounds, of which celecoxib and deracoxib are examples, can be administered parenterally as isotonic solutions in a range of solvents including polyethylene glycol and propylene glycol.

Above-cited U.S. Pat. No. 5,633,272 discloses that its subject isoxazolyl benzenesulfonamides, of which valdecoxib is an example, can be administered parenterally as isotonic solutions in a range of solvents including polyethylene glycol and propylene glycol.

Above-cited U.S. Pat. No. 5,474,995 discloses that its subject (methylsulfonyl)phenyl furanones, of which rofecoxib is an example, can be administered parenterally in an isotonic solution in 1,3-butanediol. Also disclosed therein are syrups and elixirs for oral administration, formulated with a sweetening agent such as propylene glycol.

Above-cited U.S. Pat. No. 5,861,419 discloses that its subject substituted pyridines, of which etoricoxib is an example, can be administered parenterally in an isotonic solution in 1,3-butanediol. Also disclosed therein are syrups and elixirs for oral administration, formulated with a sweetening agent such as propylene glycol.

As an alternative to directly imbibable liquid formulations of a drug, it is known to encapsulate liquid formulations, for example in soft gelatin capsules or hard gelatin capsules, to provide a discrete dosage form.

Many selective COX-2 inhibitory compounds, including celecoxib, deracoxib, valdecoxib, rofecoxib and etoricoxib, have low solubility in aqueous media. In addition, some, for example celecoxib, have relatively high dose requirements. These properties present practical problems in formulating concentrated solutions of selective COX-2 inhibitory drugs for rapid-onset, oral administration. With respect to such high dose, low solubility drugs, the size of the gelatin capsule or volume of solution required to provide a therapeutic dose becomes a limiting factor. For example, a drug that has a solubility of 10 mg/ml in a given solvent and a therapeutic dose of 400 mg/day would require ingestion of 40 ml of solution. Such a volume is inconvenient or unacceptable for consumption in imbibable form; this volume also presents particular problems where a discrete dosage form is desired because capsules that contain more than about 1.0 ml to about 1.5 ml of liquid are generally considered to be too large for comfortable consumption. Alternatively, multiple capsules would need to be ingested in order to get the required dose.

If a selective COX-2 inhibitory drug is to be formulated as a solution, highly concentrated solutions would be beneficial for several reasons. First, concentrated solutions are less costly to package and easier to transport and handle than dilute solutions. Second, as indicated above, concentrated solutions provide dose flexibility as they can be administered with or without dilution. And third, dilute drug solutions can require consumption of large volumes of fluid, which can be uncomfortable for many patient populations. For these and other reasons, therefore, if the difficulties discussed above could be overcome, it would be a much desired advance in the art to provide an effective concentrated solution formulation of a selective COX-2 inhibitory drug of low solubility, such as celecoxib, for rapid-onset indications. It would represent an especially important advance in the art to provide an effective method of treatment of acute pain, for example in headache or migraine, using such a formulation.

### SUMMARY OF THE INVENTION

According to the present invention, there is now provided an orally deliverable pharmaceutical composition comprising a selective COX-2 inhibitory drug of low water solubility, at least a substantial part, for example at least about 15% by weight, of which is in dissolved or solubilized form, in a solvent liquid comprising a pharmaceutically acceptable glycol ether.

Preferably the glycol ether conforms to formula (VII):

R¹—O—((CH₂)ₘO)ₙ—R² (VII)

wherein R¹ and R² are independently hydrogen or C₁₋₆ alkyl, C₁₋₆ alkenyl, phenyl or benzyl groups, but no more than one of R¹ and R² is hydrogen; m is an integer of 2 to about 5; and n is an integer of 1 to about 20.

Compositions of the invention are especially useful for selective COX-2 inhibitory compounds having solubility in water lower than about 1 mg/ml.

The term “solvent liquid” herein encompasses all of the components of the liquid medium in which the selective COX-2 inhibitory drug is dissolved or solubilized including but not limited to one or more solvents, co-solvents, surfactants, co-surfactants, sweeteners, flavoring agents, colorants, etc.

In a presently preferred embodiment, an orally deliverable pharmaceutical composition is provided comprising a selective COX-2 inhibitory drug of low water solubility and a solvent liquid comprising a pharmaceutically acceptable glycol ether, wherein substantially all of the drug is present in dissolved or solubilized form in the solvent liquid. In this embodiment, the solvent liquid preferably contains less than about 25% water. However, a composition of this embodiment can, if desired, be diluted with a suitable amount of water for oral administration.

In another embodiment, a composition of the invention comprises, in addition to a first portion of the drug in dissolved or solubilized form, a second portion of the drug in particulate form dispersed in the solvent liquid. In this embodiment, part of the drug is in solution and part is in suspension. Such a composition of a selective COX-2 inhibitory drug dissolved in part and dispersed in part in a solvent liquid is referred to herein as a “solution/suspension”.

In a presently preferred embodiment, the solution or solution/suspension is encapsulated in one or more capsules that release the drug within a short period of time after entry into the gastrointestinal tract. The preferred encapsulation material is gelatin; however, other materials can be used. The particular mechanism of drug release is not important and can include such mechanisms as erosion, degradation, dissolution, etc. In this embodiment, each capsule preferably contains about 0.3 ml to about 1.8 ml (about 5 minim to about 30 minim) of solution or solution/suspension and contains a therapeutically effective amount of the selective COX-2 inhibitory drug.

Compositions of the invention have been found to resolve at least some of the difficulties alluded to above in a surprisingly effective manner. Thus, for the first time, a selective COX-2 inhibitory drug of low water solubility is presented in concentrated solution in a convenient dosage form for oral administration. A particular advantage of formulations of the invention is that following oral administration thereof, the drug is rapidly absorbed into the bloodstream. By virtue of this rapid absorption, formulations of the invention can provide rapid onset of therapeutic effect.

It can be theorized that a poorly water-soluble selective COX-2 inhibitory drug such as celecoxib can provide more rapid onset of therapeutic effect when orally administered in solution than in particulate form because the process of dissolution in the gastrointestinal tract is not required. An even greater advantage by comparison with a solid formulation can be postulated because neither disintegration nor dissolution is required in the case of the solution composition.

Additionally, a drug administered in solution can be available for absorption higher in the alimentary tract, for example, in the mouth and esophagus, than one that becomes available for absorption only upon disintegration of the carrier formulation in the stomach or bowel.

A further advantage of solutions and other liquid dosage forms for many patients is that they are easy to swallow. A yet further advantage of imbibable liquid dosage forms such as solutions is that metering of doses is continuously variable, providing infinite dose flexibility. The benefits of ease of swallowing and dose flexibility are particularly advantageous for infants, children and the elderly.

When encapsulated, a solution or solution/suspension can provide the subject with the beneficial rapid absorption characteristics associated with liquid formulations in addition to the convenience of a discrete, easy to swallow capsule form.

Also provided by the present invention are methods for preparation of and methods for therapeutic and/or prophylactic use of compositions of the present invention.

In one embodiment, a method of analgesia is provided comprising orally administering, to a subject in need of analgesia, an effective pain-relieving amount of an aminosulfonyl-comprising selective COX-2 inhibitory drug composition of the invention. In another embodiment, a method of treatment and/or prevention of headache or migraine is provided comprising orally administering, to a subject in need of such treatment or prevention, an aminosulfonyl-comprising selective COX-2 inhibitory drug composition of the invention and a vasomodulator, for example a methylxanthine, wherein the selective COX-2 inhibitory drug and the vasomodulator are administered in effective pain-relieving total and relative amounts. The selective COX-2 inhibitory drug and the vasomodulator can be administered as components of separate compositions or of a single composition. Such a single composition comprising (a) an aminosulfonyl-comprising selective COX-2 inhibitory drug, formulated as provided herein, and (b) a vasomodulator, is a further embodiment of the invention. A presently preferred methylxanthine is caffeine.

Other features of this invention will be in part apparent and in part pointed out hereinafter.

### BRIEF DESCRIPTION OF THE DRAWINGS

FIG. 1 shows the blood plasma concentrations of two formulations of celecoxib, F1 and a solid capsule formulation, after administration to dogs. The composition of the F1 formulation is shown in Table 2 herein.

FIG. 2 shows the blood plasma concentrations of two formulations of celecoxib, F3 and a solid capsule formulation, after administration to dogs. The composition of the F3 formulation is shown in Table 2 herein.

FIG. 3 shows the blood plasma concentrations of two formulations of celecoxib, F4 and a solid capsule formulation, after administration to dogs. The composition of the F4 formulation is shown in Table 2 herein.

FIG. 4 shows the in vitro dissolution profiles of five formulations: F1, F3, F4, F5 and F7. Compositions of these formulations are described in Table 2 herein.

FIG. 5 shows the in vitro dissolution profiles of three formulations: F8, F9 and F10. Compositions of these formulations are described in Table 2 herein.

### DETAILED DESCRIPTION OF THE INVENTION

The present invention provides pharmaceutical compositions and dosage forms thereof suitable for oral administration, the compositions comprising a selective COX-2 inhibitory drug of low solubility in water.

Any such selective COX-2 inhibitory drug known in the art can be used, including without limitation compounds disclosed in the patents and publications listed below, each of which is individually incorporated herein by reference.

U.S. Pat. No. 5,344,991 to Reitz & Li.

U.S. Pat. No. 5,380,738 to Norman et al.

U.S. Pat. No. 5,393,790 to Reitz et al.

U.S. Pat. No. 5,401,765 to Lee.

U.S. Pat. No. 5,418,254 to Huang & Reitz.

U.S. Pat. No. 5,420,343 to Koszyk & Weier.

U.S. Pat. No. 5,434,178 to Talley & Rogier.

U.S. Pat. No. 5,436,265 to Black et al.

Above-cited U.S. Pat. No. 5,466,823.

Above-cited U.S. Pat. No. 5,474,995.

U.S. Pat. No. 5,475,018 to Lee & Bertenshaw.

U.S. Pat. No. 5,486,534 to Lee et al.

U.S. Pat. No. 5,510,368 to Lau et al.

U.S. Pat. No. 5,521,213 to Prasit et al.

U.S. Pat. No. 5,536,752 to Ducharme et al.

U.S. Pat. No. 5,543,297 to Cromlish et al.

U.S. Pat. No. 5,547,975 to Talley et al.

U.S. Pat. No. 5,550,142 to Ducharme et al.

U.S. Pat. No. 5,552,422 to Gauthier et al.

U.S. Pat. No. 5,585,504 to Desmond et al.

U.S. Pat. No. 5,593,992 to Adams et al.

U.S. Pat. No. 5,596,008 to Lee.

U.S. Pat. No. 5,604,253 to Lau et al.

U.S. Pat. No. 5,604,260 to Guay & Li.

U.S. Pat. No. 5,616,458 to Lipsky et al.

U.S. Pat. No. 5,616,601 to Khanna et al.

U.S. Pat. No. 5,620,999 to Weier et al.

Above-cited U.S. Pat. No. 5,633,272.

U.S. Pat. No. 5,639,780 to Lau et al.

U.S. Pat. No. 5,643,933 to Talley et al.

U.S. Pat. No. 5,658,903 to Adams et al.

U.S. Pat. No. 5,668,161 to Talley et al.

U.S. Pat. No. 5,670,510 to Huang & Reitz.

U.S. Pat. No. 5,677,318 to Lau.

U.S. Pat. No. 5,681,842 to Dellaria & Gane.

U.S. Pat. No. 5,686,460 to Nicolaï et al.

U.S. Pat. No. 5,686,470 to Weier et al.

U.S. Pat. No. 5,696,143 to Talley et al.

U.S. Pat. No. 5,710,140 to Ducharme et al.

U.S. Pat. No. 5,716,955 to Adams et al.

U.S. Pat. No. 5,723,485 to Güngör & Teulon.

U.S. Pat. No. 5,739,166 to Reitz et al.

U.S. Pat. No. 5,741,798 to Lazer et al.

U.S. Pat. No. 5,756,499 to Adams et al.

U.S. Pat. No. 5,756,529 to Isakson & Talley.

U.S. Pat. No. 5,776,967 to Kreft et al.

U.S. Pat. No. 5,783,597 to Beers & Wachter.

U.S. Pat. No. 5,789,413 to Black et al.

U.S. Pat. No. 5,807,873 to Nicolaï & Teulon.

U.S. Pat. No. 5,817,700 to Dube et al.

U.S. Pat. No. 5,830,911 to Failli et al.

U.S. Pat. No. 5,849,943 to Atkinson & Wang.

U.S. Pat. No. 5,859,036 to Sartori et al.

Above-cited U.S. Pat. No. 5,861,419.

U.S. Pat. No. 5,866,596 to Sartori & Teulon.

U.S. Pat. No. 5,869,524 to Failli.

U.S. Pat. No. 5,869,660 to Adams et al.

U.S. Pat. No. 5,883,267 to Rossen et al.

U.S. Pat. No. 5,892,053 to Zhi et al.

U.S. Pat. No. 5,922,742 to Black et al.

U.S. Pat. No. 5,929,076 to Adams & Garigipati.

U.S. Pat. No. 5,932,598 to Talley et al.

U.S. Pat. No. 5,935,990 to Khanna et al.

U.S. Pat. No. 5,945,539 to Haruta et al.

U.S. Pat. No. 5,958,978 to Yamazaki et al.

U.S. Pat. No. 5,968,958 to Guay et al.

U.S. Pat. No. 5,972,950 to Nicolaï & Teulon.

U.S. Pat. No. 5,973,191 to Marnett & Kalgutkar.

Above-cited U.S. Pat. No. 5,981,576.

U.S. Pat. No. 5,994,381 to Haruta et al.

U.S. Pat. No. 6,002,014 to Haruta et al.

U.S. Pat. No. 6,004,960 to Li et al.

U.S. Pat. No. 6,005,000 to Hopper et al.

U.S. Pat. No. 6,020,343 to Belley et al.

U.S. Pat. No. 6,020,347 to DeLaszlo & Hagmann.

Above-cited U.S. Pat. No. 6,034,256.

U.S. Pat. No. 6,040,319 to Corley et al.

U.S. Pat. No. 6,040,450 to Davies et al.

U.S. Pat. No. 6,046,208 to Adams et al.

U.S. Pat. No. 6,046,217 to Friesen et al.

U.S. Pat. No. 6,057,319 to Black et al.

U.S. Pat. No. 6,063,804 to De Nanteuil et al.

U.S. Pat. No. 6,063,807 to Chabrier de Lassauniere & Broquet.

U.S. Pat. No. 6,071,954 to LeBlanc et al.

U.S. Pat. No. 6,077,868 to Cook et al.

U.S. Pat. No. 6,077,869 to Sui & Wachter.

U.S. Pat. No. 6,083,969 to Ferro et al.

U.S. Pat. No. 6,096,753 to Spohr et al.

U.S. Pat. No. 6,133,292 to Wang et al.

International Patent Publication No. WO 94/15932.

International Patent Publication No. WO 96/19469.

International Patent Publication No. WO 96/26921.

International Patent Publication No. WO 96/31509.

International Patent Publication No. WO 96/36623.

International Patent Publication No. WO 96/38418.

International Patent Publication No. WO 97/03953.

International Patent Publication No. WO 97/10840.

International Patent Publication No. WO 97/13755.

International Patent Publication No. WO 97/13767.

International Patent Publication No. WO 97/25048.

International Patent Publication No. WO 97/30030.

International Patent Publication No. WO 97/34882.

International Patent Publication No. WO 97/46524.

International Patent Publication No. WO 98/04527.

International Patent Publication No. WO 98/06708.

International Patent Publication No. WO 98/07425.

International Patent Publication No. WO 98/17292.

International Patent Publication No. WO 98/21195.

International Patent Publication No. WO 98/22457.

International Patent Publication No. WO 98/32732.

International Patent Publication No. WO 98/41516.

International Patent Publication No. WO 98/43966.

International Patent Publication No. WO 98/45294.

International Patent Publication No. WO 98/47871.

International Patent Publication No. WO 99/01130.

International Patent Publication No. WO 99/01131.

International Patent Publication No. WO 99/01452.

International Patent Publication No. WO 99/01455.

International Patent Publication No. WO 99/10331.

International Patent Publication No. WO 99/10332.

International Patent Publication No. WO 99/11605.

International Patent Publication No. WO 99/12930.

International Patent Publication No. WO 99/14195.

International Patent Publication No. WO 99/14205.

International Patent Publication No. WO 99/15505.

International Patent Publication No. WO 99/23087.

International Patent Publication No. WO 99/24404.

International Patent Publication No. WO 99/25695.

International Patent Publication No. WO 99/35130.

International Patent Publication No. WO 99/61016.

International Patent Publication No. WO 99/61436.

International Patent Publication No. WO 99/62884.

International Patent Publication No. WO 99/64415.

International Patent Publication No. WO 00/01380.

International Patent Publication No. WO 00/08024.

International Patent Publication No. WO 00/10993.

International Patent Publication No. WO 00/13684.

International Patent Publication No. WO 00/18741.

International Patent Publication No. WO 00/18753.

International Patent Publication No. WO 00/23426.

Above-cited International Patent Publication No. WO 00/24719.

International Patent Publication No. WO 00/26216.

International Patent Publication No. WO 00/31072.

International Patent Publication No. WO 00/40087.

International Patent Publication No. WO 00/56348.

European Patent Application No. 0 799 823.

European Patent Application No. 0 846 689.

Above-cited European Patent Application No. 0 863 134.

European Patent Application No. 0 985 666.

Compositions of the invention are especially useful for compounds having the formula (VIII):

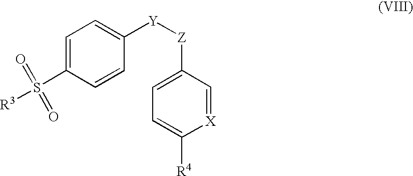

where R³ is a methyl or amino group, R⁴ is hydrogen or a C₁₋₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups. Preferred such five- to six-membered rings are cyclopentenone, furanone, methylpyrazole, isoxazole and pyridine rings substituted at no more than one position.

Illustratively, celecoxib, deracoxib, valdecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid and 2-(3,4-difluorophenyl)-4-(3-hydroxy-3-methyl-1-butoxy)-5-[4-(methylsulfonyl)phenyl]-3-(2H)-pyridazinone, more particularly celecoxib, valdecoxib, rofecoxib and etoricoxib, and still more particularly celecoxib and valdecoxib, are useful in the method and composition of the invention.

The invention is illustrated herein with particular reference to celecoxib, and it will be understood that any other selective COX-2 inhibitory drug of low solubility in water can, if desired, be substituted in whole or in part for celecoxib in compositions herein described. For example, compositions of the invention are suitable for formulation of valdecoxib, alone or in combination with celecoxib.

Celecoxib compositions of the invention exhibit improved performance as selective COX-2 inhibitory medications. In particular, these compositions provide celecoxib to a patient at a dose and release rate sufficient to enable rapid-onset inhibition of COX-2.

Celecoxib used in pharmaceutical compositions of the present invention can be prepared by any known manner, for example in the manner set forth in above-cited U.S. Pat. No. 5,466,823 or in above-cited U.S. Pat. No. 5,892,053. Other selective COX-2 inhibitory drugs can be prepared by any known manner, including the manner set forth in patent publications disclosing such drugs; for example in the case of valdecoxib in above-cited U.S. Pat. No. 5,633,272, and in the case of rofecoxib in above-cited U.S. Pat. No. 5,474,995.

Celecoxib compositions of the present invention preferably comprise celecoxib in a daily dosage amount of about 50 mg to about 1000 mg, more preferably about 75 mg to about 400 mg, and most preferably about 100 mg to about 200 mg.

For other selective COX-2 inhibitory drugs, a daily dosage amount can be in a range known to be therapeutically effective for such drugs. Preferably, the daily dosage amount is in a range providing therapeutic equivalence to celecoxib in the daily dose ranges indicated immediately above.

Compositions of the present invention are preferably in the form of a concentrated solution that may or may not be encapsulated as a discrete article. If encapsulated, preferably a single such article or a small plurality (up to about 10, more preferably no more than about 4) of such articles is sufficient to provide the daily dose. Alternatively, compositions of the present invention are in the form of a concentrated imbibable liquid. The phrase “imbibable liquid” is used herein to refer to an unencapsulated homogeneous flowable mass, such as a solution or solution/suspension, administered orally and swallowed in liquid form and from which single dosage units are measurably removable.

Dosage units of celecoxib compositions of the invention typically contain about 10 mg to about 400 mg of celecoxib, for example, a 10, 20, 37.5, 50, 75, 100, 125, 150, 175, 200, 250, 300, 350, or 400 mg dose of celecoxib. Preferred dosage units contain about 50 mg to about 400 mg of celecoxib. More preferred dosage unit forms contain about 100 mg to about 200 mg of celecoxib. A particular dosage unit can be selected to accommodate the desired frequency of administration used to achieve a specified daily dose. For example, a daily dosage amount of 400 mg can be accommodated by administration of one 200 mg dosage unit, or two 100 mg dosage units, twice a day. The amount of the unit dosage form of the composition that is administered and the dosage regimen for treating the condition or disorder will depend on a variety of factors, including the age, weight, sex and medical condition of the subject, the severity of the condition or disorder, the route and frequency of administration, and the particular selective COX-2 inhibitory drug selected, and thus may vary widely. It is contemplated, however, that for most purposes a once-a-day or twice-a-day administration regimen provides the desired therapeutic efficacy.

In a celecoxib composition, celecoxib can be present in the composition at a minimum concentration of about 1%, preferably about 4%, more preferably about 10%, and still more preferably about 20%, by weight. Where the selective COX-2 inhibitory drug is therapeutically effective at lower doses than celecoxib, the minimum concentration can be lower than that indicated immediately above for celecoxib; for example in the case of valdecoxib the drug can be present at a minimum concentration of about 0.1% by weight. The maximum concentration is dictated in part by solubility of the drug in the solvent liquid; it is contemplated that, where a portion of the drug is suspended in particulate form in the solvent liquid, the maximum concentration can be about 75% by weight or higher. In a composition having substantially all of the drug in dissolved or solubilized form, it is contemplated that the maximum concentration can be about 50% by weight or higher, but more typically the maximum concentration is about 35% by weight.

Compositions of the invention are useful in treatment and prevention of a very wide range of disorders mediated by COX-2, including but not restricted to disorders characterized by inflammation, pain and/or fever. Such compositions are especially useful as anti-inflammatory agents, such as in treatment of arthritis, with the additional benefit of having significantly less harmful side effects than compositions of conventional nonsteroidal anti-inflammatory drugs (NSAIDs) that lack selectivity for COX-2 over COX-1. In particular, compositions of the invention have reduced potential for gastrointestinal toxicity and gastrointestinal irritation including upper gastrointestinal ulceration and bleeding, reduced potential for renal side effects such as reduction in renal function leading to fluid retention and exacerbation of hypertension, reduced effect on bleeding times including inhibition of platelet function, and possibly a lessened ability to induce asthma attacks in aspirin-sensitive asthmatic subjects, by comparison with compositions of conventional NSAIDs. Thus compositions of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendinitis, bursitis, allergic neuritis, cytomegalovirus infectivity, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation including that following ophthalmic surgery such as cataract surgery or refractive surgery.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic diseases, such as retinitis, conjunctivitis, retinopathies, uveitis, ocular photophobia, and of acute injury to the eye tissue.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are useful in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, bums, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemaginomas, angiofibroma of the nasopharynx and avascular necrosis of bone; and disorders of the female reproductive system such as endometriosis.

Such compositions are useful in prevention and treatment of benign and malignant tumors and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in patients at risk of FAP.

Such compositions inhibit prostanoid-induced smooth muscle contraction by inhibiting synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Because of the rapid onset of therapeutic effect that can be exhibited by compositions of the invention, these compositions have particular advantages over prior formulations for treatment of acute COX-2 mediated disorders, especially for relief of pain, for example in headache, including sinus headache and migraine.

Preferred uses for compositions of the present invention are for treatment of rheumatoid arthritis and osteoarthritis, for pain management generally (particularly post-oral surgery pain, post-general surgery pain, post-orthopedic surgery pain, and acute flares of osteoarthritis), for prevention and treatment of headache and migraine, for treatment of Alzheimer's disease, and for colon cancer chemoprevention.

For treatment of rheumatoid arthritis or osteoarthritis, compositions of the invention can be used to provide a daily dose of celecoxib of about 50 mg to about 1000 mg, preferably about 100 mg to about 600 mg, more preferably about 150 mg to about 500 mg, still more preferably about 175 mg to about 400 mg, for example about 200 mg. A daily dose of celecoxib of about 0.7 to about 13 mg/kg body weight, preferably about 1.3 to about 8 mg/kg body weight, more preferably about 2 to about 6.7 mg/kg body weight, and still more preferably about 2.3 to about 5.3 mg/kg body weight, for example about 2.7 mg/kg body weight, is generally appropriate when administered in a composition of the invention. The daily dose can be administered in one to about four doses per day, preferably one or two doses per day.

For treatment of Alzheimer's disease or cancer, compositions of the invention can be used to provide a daily dose of celecoxib of about 50 mg to about 1000 mg, preferably about 100 mg to about 800 mg, more preferably about 150 mg to about 600 mg, and still more preferably about 175 mg to about 400 mg, for example about 400 mg. A daily dose of about 0.7 to about 13 mg/kg body weight, preferably about 1.3 to about 10.7 mg/kg body weight, more preferably about 2 to about 8 mg/kg body weight, and still more preferably about 2.3 to about 5.3 mg/kg body weight, for example about 5.3 mg/kg body weight, is generally appropriate when administered in a composition of the invention. The daily dose can be administered in one to about four doses per day, preferably one or two doses per day.

For pain management generally and specifically for treatment and prevention of headache and migraine, compositions of the invention can be used to provide a daily dose of celecoxib of about 50 mg to about 1000 mg, preferably about 100 mg to about 600 mg, more preferably about 150 mg to about 500 mg, and still more preferably about 175 mg to about 400 mg, for example about 200 mg. A daily dose of celecoxib of about 0.7 to about 13 mg/kg body weight, preferably about 1.3 to about 8 mg/kg body weight, more preferably about 2 to about 6.7 mg/kg body weight, and still more preferably about 2.3 to about 5.3 mg/kg body weight, for example about 2.7 mg/kg body weight, is generally appropriate when administered in a composition of the invention. The daily dose can be administered in one to about four doses per day. Administration at a rate of one 50 mg dose unit four times a day, one 100 mg dosage unit or two 50 mg dose units twice a day or one 200 mg dosage unit, two 100 mg dosage units or four 50 mg dosage units once a day is preferred.

For selective COX-2 inhibitory drugs other than celecoxib, appropriate doses can be selected by reference to the patent literature cited hereinabove.

Besides being useful for human treatment, compositions of the invention are also useful for veterinary treatment of companion animals, exotic animals, farm animals, and the like, particularly mammals including rodents. More particularly, compositions of the invention are useful for veterinary treatment of COX-2 mediated disorders in horses, dogs and cats.

The present invention also is directed to a therapeutic method of treating a condition or disorder where treatment with a COX-2 inhibitor is indicated, the method comprising oral administration of one or more pharmaceutical compositions of the present invention to a patient in need thereof. The dosage regimen to prevent, give relief from, or ameliorate the condition or disorder preferably corresponds to once-a-day or twice-a-day treatment, but can be modified in accordance with a variety of factors. These include the type, age, weight, sex, diet and medical condition of the patient and the nature and severity of the disorder. Thus, the dosage regimen actually employed can vary widely and can therefore deviate from the preferred dosage regimens set forth above.

Initial treatment of a patient suffering from a condition or disorder where treatment with a COX-2 inhibitor is indicated can begin with a dosage regimen as indicated above. Treatment is generally continued as necessary over a period of several weeks to several months or years until the condition or disorder has been controlled or eliminated. Patients undergoing treatment with a composition of the invention can be routinely monitored by any of the methods well known in the art to determine the effectiveness of therapy. Continuous analysis of data from such monitoring permits modification of the treatment regimen during therapy so that optimally effective amounts of drug are administered at any point in time, and so that the duration of treatment can be determined. In this way, the treatment regimen and dosing schedule can be rationally modified over the course of therapy so that the lowest amount of celecoxib exhibiting satisfactory effectiveness is administered, and so that administration is continued only for so long as is necessary to successfully treat the condition or disorder.

The present compositions can be used in combination therapies with opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise use of a composition of the invention with one or more compounds selected from aceclofenac, acemetacin, e-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lornoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac (see The Merck Index, 12th Edition (1996), Therapeutic Category and Biological Activity Index, lists therein headed “Analgesic”, “Anti-inflammatory” and “Antipyretic”).

Particularly preferred combination therapies comprise use of a celecoxib composition of the invention with an opioid compound, more particularly where the opioid compound is codeine, meperidine, morphine or a derivative thereof.

The compound to be administered in combination with celecoxib can be formulated separately from the celecoxib or co-formulated with the celecoxib in a composition of the invention. Where celecoxib is co-formulated with a second drug, for example an opioid drug, the second drug can be formulated in immediate-release, rapid-onset, sustained-release or dual-release form.

In an embodiment of the invention, particularly where the COX-2 mediated condition is headache or migraine, the present selective COX-2 inhibitory drug composition is administered in combination therapy with a vasomodulator, preferably a xanthine derivative having vasomodulatory effect, more preferably an alkylxanthine compound.

Combination therapies wherein an alkylxanthine compound is co-administered with a selective COX-2 inhibitory drug composition as provided herein are embraced by the present embodiment of the invention whether or not the alkylxanthine is a vasomodulator and whether or not the therapeutic effectiveness of the combination is to any degree attributable to a vasomodulatory effect. The term “alkylxanthine” herein embraces xanthine derivatives having one or more C₁₋₄ alkyl, preferably methyl, substituents, and pharmaceutically acceptable salts of such xanthine derivatives. Dimethylxanthines and trimethylxanthines, including caffeine, theobromine and theophylline, are especially preferred. Most preferably, the alkylxanthine compound is caffeine.

The total and relative dosage amounts of the selective COX-2 inhibitory drug and of the vasomodulator or alkylxanthine are selected to be therapeutically and/or prophylactically effective for relief of pain associated with the headache or migraine. Suitable dosage amounts will depend on the particular selective COX-2 inhibitory drug and the particular vasomodulator or alkylxanthine selected. For example, in a combination therapy with celecoxib and caffeine, typically the celecoxib will be administered in a daily dosage amount of about 50 mg to about 1000 mg, preferably about 100 mg to about 600 mg, and the caffeine in a daily dosage amount of about 1 mg to about 500 mg, preferably about 10 mg to about 400 mg, more preferably about 20 mg to about 300 mg.

The vasomodulator or alkylxanthine component of the combination therapy can be administered in any suitable dosage form by any suitable route, preferably orally. The vasomodulator or alkylxanthine can optionally be coformulated with the selective COX-2 inhibitory drug in a single oral dosage form. Thus a solution or solution/suspension formulation of the invention optionally comprises both an aminosulfonyl-comprising selective COX-2 inhibitory drug and a vasomodulator or alkylxanthine such as caffeine, in total and relative amounts consistent with the dosage amounts set out hereinabove.

The phrase “in total and relative amounts effective to relieve pain”, with respect to amounts of a selective COX-2 inhibitory drug and a vasomodulator or alkylxanthine in a composition of the present embodiment, means that these amounts are such that (a) together these components are effective to relieve pain, and (b) each component is or would be capable of contribution to a pain-relieving effect if the other component is or were not present in so great an amount as to obviate such contribution.

Compositions of the present invention comprise celecoxib and/or another selective COX-2 inhibitory drug of low solubility in a solvent liquid suitable for oral administration. The solvent liquid comprises a pharmaceutically acceptable glycol ether and optional additional components, including wetting agents, suspending agents, flocculating agents, buffers, co-solvents, colorants, sweeteners and flavoring agents, among others. Such optional additional components must be physically and chemically compatible with the other ingredients of the composition and must not be deleterious to the recipient. Importantly, some of the above-listed classes of excipients overlap each other. Compositions of the present invention can be adapted for administration by any suitable oral route by selection of appropriate solvent liquid components and a dose of the drug effective for the treatment intended. Accordingly, components employed in the solvent liquid can themselves be solids or liquids, or both.

An imbibable celecoxib composition of the invention can be in the form of, for example, a solution, a solution/suspension, an elixir, a syrup, or any other liquid form reasonably adapted for oral administration. Such compositions can also comprise excipients selected from, for example, wetting agents, emulsifying and suspending agents, sweetening and flavoring agents, surfactants and co-surfactants.

Alternatively, a composition of the present invention can made in the form of discrete unit dosage articles, for example, soft or hard gelatin or hydroxypropylmethylcellulose (HPMC) capsules, each containing a predetermined amount of celecoxib in a solvent liquid.

Compositions of the invention can be prepared by any suitable method of pharmacy that includes the step of bringing into association the selective COX-2 inhibitory drug and the solvent liquid. In general, celecoxib compositions are prepared by uniformly and intimately admixing celecoxib with a solvent liquid and then, if desired, encapsulating the resulting solution or solution/suspension, preferably in a soft gelatin capsule. Encapsulation can be performed by any method known in the art including, but not limited to, the plate process and the rotary die process as described, for example, by Ansel et al. (1995) in Pharmaceutical Dosage Forms and Drug Delivery Systems, 6th ed., Williams & Wilkins, Baltimore, Md., pp. 176-182.

An embodiment of the present invention is a composition comprising a therapeutically effective amount of a selective COX-2 inhibitory drug of low solubility, for example celecoxib, fully dissolved or solubilized in a solvent liquid comprising a pharmaceutically acceptable glycol ether. In this embodiment, substantially no part of the drug is suspended in particulate form in the solvent liquid. Compositions of this embodiment can be formulated either in an imbibable or discrete dosage form.

Glycol ethers useful as solvents in the present invention preferably conform to formula (VII):

R¹—O—((CH₂)ₘO)ₙ—R² (VII)

wherein R¹ and R² are independently hydrogen or C₁₋₆ alkyl, C₁₋₆ alkenyl, phenyl or benzyl groups, but no more than one of R¹ and R² is hydrogen; m is an integer of 2 to about 5; and n is an integer of 1 to about 20. It is preferred that one of R¹ and R² is a C₁₋₄ alkyl group and the other is hydrogen or a C₁₋₄ alkyl group; more preferably at least one of R¹ and R² is a methyl or ethyl group. It is preferred that m is 2. It is preferred that n is an integer of 1 to about 4, more preferably 2.

Glycol ethers used in compositions of the present invention typically have a molecular weight of about 75 to about 1000, preferably about 75 to about 500, and more preferably about 100 to about 300. Importantly, the glycol ethers used in compositions of the present invention must be pharmaceutically acceptable and must meet all other conditions prescribed herein.

Non-limiting examples of glycol ethers that may be used in compositions of the present invention include ethylene glycol monomethyl ether, ethylene glycol dimethyl ether, ethylene glycol monoethyl ether, ethylene glycol diethyl ether, ethylene glycol monobutyl ether, ethylene glycol dibutyl ether, ethylene glycol monophenyl ether, ethylene glycol monobenzyl ether, ethylene glycol butylphenyl ether, ethylene glycol terpinyl ether, diethylene glycol monomethyl ether, diethylene glycol dimethyl ether, diethylene glycol monoethyl ether, diethylene glycol diethyl ether, diethylene glycol divinyl ether, ethylene glycol monobutyl ether, diethylene glycol dibutyl ether, diethylene glycol monoisobutyl ether, triethylene glycol dimethyl ether, triethylene glycol monoethyl ether, triethylene glycol monobutyl ether, tetraethylene glycol dimethyl ether, and mixtures thereof. See for example Flick (1998): Industrial Solvents Handbook, 5th ed., Noyes Data Corporation, Westwood, N.J. A presently preferred glycol ether solvent is diethylene glycol monoethyl ether, sometimes referred to in the art as DGME or ethoxydiglycol. It is available for example under the trademark Transcutol™ of Gattefossé Corporation.

Compositions of the present invention optionally comprise one or more pharmaceutically acceptable co-solvents. Non-limiting examples of co-solvents suitable for use in compositions of the present invention include any glycol ether listed above; alcohols, for example ethanol and n-butanol; glycols not listed above, for example propylene glycol, 1,3-butanediol and polyethylene glycol such as PEG-400; oleic and linoleic acid triglycerides, for example soybean oil; caprylic/capric triglycerides, for example Miglyol™ 812 of Huls; caprylic/capric mono- and diglycerides, for example Capmul™ MCM of Abitec; polyoxyethylene caprylic/capric glycerides such as polyoxyethylene (8) caprylic/capric mono- and diglycerides, for example Labrasol™ of Gattefossé; propylene glycol fatty acid esters, for example propylene glycol laurate; polyoxyethylene (35) castor oil, for example Cremophor™ EL of BASF; polyoxyethylene glyceryl trioleate, for example Tagat™ TO of Goldschmidt; and lower alkyl esters of fatty acids, for example ethyl butyrate, ethyl caprylate and ethyl oleate.

Many co-solvents useful in compositions of the present invention, including some of those listed above, have surfactant properties. Without being bound by theory, it is believed that certain compositions having surfactants and co-surfactants self-emulsify in the aqueous environment of the gastrointestinal tract. Preferably, surfactants and co-surfactants are selected so as to form in the gastrointestinal tract microemulsions, wherein the size of the emulsion droplets is less than about 200 nm. An illustrative preferred solvent liquid comprises diethylene glycol monoethyl ether as solvent together with polyoxyethylene glyceryl trioleate and caprylic/capric mono- and diglycerides as co-solvents.

Concentrated solution compositions of the invention preferably contain less than about 25% water. More preferably less than about 10% water is present, and most preferably no substantial amount of water is present, in a concentrated solution composition of the invention. The presence of water greatly reduces the solubility of the drug in the solvent liquid, and as a consequence seriously limits the maximum concentration at which the solution composition can be prepared. In the case of solution/suspension compositions, greater amounts of water can generally be tolerated; indeed in one embodiment of the invention the relative amounts of the drug in solution and in suspension are controlled by addition of water to reduce solubility.

Compositions of this embodiment optionally contain pharmaceutically acceptable excipients such as sweeteners, antioxidants, preservatives, etc. Through selection and combination of excipients, compositions can be provided exhibiting improved performance with respect to solvent liquid concentration, dissolution, efficacy, flavor and overall patient compliance.

Compositions of the present invention optionally comprise one or more pharmaceutically acceptable sweeteners. Non-limiting examples of sweeteners that can be used include mannitol, propylene glycol, sodium saccharin, acesulfame K, neotame and aspartame. Alternatively or in addition, a viscous sweetener such as sorbitol solution, syrup (sucrose solution) or high-fructose corn syrup can be used and, in addition to sweetening effects, can also be useful to increase viscosity or to retard sedimentation.

Compositions of the present invention optionally comprise one or more pharmaceutically acceptable antioxidants. Non-limiting examples of antioxidants that can be used include ascorbic acid, sodium ascorbate, ascorbic acid palmitate, fumaric acid, malic acid, α-tocopherol, butylated hydroxyanisole, propyl gallate and sodium metabisulfite.

Compositions of the present invention optionally comprise one or more pharmaceutically acceptable preservatives other than the antioxidants listed above. Non-limiting examples of such preservatives include benzalkonium chloride, benzethonium chloride, benzyl alcohol, chlorobutanol, phenol, phenylethyl alcohol, phenylmercuric nitrate and thimerosal.

Additionally, compositions of the present invention optionally comprise one or more pharmaceutically acceptable buffering agents, flavoring agents, colorants, stabilizers and/or thickeners. Buffers can be used to control pH of the formulation and can thereby modulate drug solubility. Flavoring agents can enhance patient compliance by making the composition more palatable, and colorants can provide a product with a more aesthetic and/or distinctive appearance. Non-limiting examples of colorants that can be used in compositions of the present invention include D&C Red No. 33, FD&C Red No. 3, FD&C Red No. 40, D&C Yellow No. 10, and C Yellow No. 6.

Some solvent liquids are suitable to maintain enough of a selective COX-2 inhibitory drug in solution to provide a therapeutically effective rapid-onset dose while also maintaining a portion of the drug undissolved but in suspension. The suspended portion typically provides less immediate release of the drug and so can extend the duration of therapeutic effect, although such extended duration is not a requirement of this embodiment of the invention.

Therefore, an embodiment of the present invention is a composition comprising a therapeutically effective amount of a selective COX-2 inhibitory drug of low solubility, for example celecoxib, in part dissolved and in part dispersed in a solvent liquid comprising a pharmaceutically acceptable glycol ether. In this embodiment, part of the drug is in solution and part is in suspension. Preferably, the solvent liquid is selected such that at least about 15% of the drug is dissolved or solubilized in the solvent liquid. As indicated above, one way of modifying a solvent liquid to increase the amount of the drug in suspension as opposed to solution is to add water in the amount necessary to give the required reduction in solubility of the drug in the solvent liquid.

Depending on the relative importance of rapid onset and sustained action for the indication for which the drug is being administered, the relative proportions of dissolved and suspended drug can be varied significantly. For example, for acute pain indications, about 50% of the drug can be in solution and about 50% of the drug can be dispersed in particulate form. Alternatively, for indications demanding longer acting therapeutic effectiveness, illustratively about 20% of the drug can be dissolved and about 80% of the drug can be dispersed in particulate form.

The particulate form of the drug can be generated mechanically, for example by milling or grinding, or by precipitation from solution. Particles formed directly from such processes are described herein as “primary particles” and can agglomerate to form secondary aggregate particles. The term “particle size” as used herein refers to size, in the longest dimension, of primary particles, unless the context demands otherwise. Particle size is believed to be an important parameter affecting the clinical effectiveness of celecoxib and other selective COX-2 inhibitory drugs of low water solubility.

Particle size can be expressed as the percentage of total particles that have a diameter smaller than a given reference diameter. For example, a useful parameter is “D₉₀ particle size”. By definition, in a batch of a drug that has a D₉₀ particle size of 60 μm, 90% of the particles have a diameter less than 60 μm.

Compositions of this embodiment of the present invention have a distribution of suspended celecoxib particle sizes such that D₉₀ of the particles, in their longest dimension, is less than about 200 μm, preferably less than about 75 μm, and more preferably less than about 25 μm. A decrease in particle size of celecoxib in accordance with this embodiment of the invention generally improves the bioavailability of the celecoxib. In addition or alternatively, suspended celecoxib particles in a composition of the invention preferably have a mean particle size less than about 10 μm, preferably about 0.1 μm to about 10 μm, for example about 1 μm.

Compositions of this embodiment can be formulated either in an imbibable or discrete dosage form. Solvents, co-solvents, sweeteners, antioxidants, preservatives, etc. can be selected as described above. Further, additional types of excipients can be useful in solution/suspension compositions, such as wetting agents, suspending agents and flocculating agents. Through selection and combination of excipients, solution/suspension compositions can be provided exhibiting improved performance with respect to drug concentration, physical stability, efficacy, flavor, and overall patient compliance.

Solution/suspension compositions of the present invention optionally comprise one or more pharmaceutically acceptable wetting agents. Surfactants, hydrophilic polymers and certain clays can be useful as wetting agents to aid in the dispersion of a hydrophobic drug such as celecoxib. Non-limiting examples of surfactants that can be used as wetting agents in compositions of the present invention include benzalkonium chloride, benzethonium chloride, cetylpyridinium chloride, dioctyl sodium sulfosuccinate, nonoxynol 9, nonoxynol 10, octoxynol 9, poloxamers (polyoxyethylene polyoxypropylene block copolymers), polyoxyethylene (8) caprylic/capric mono- and diglycerides (e.g., Labrasol™ of Gattefossé), polyoxyethylene (35) castor oil, polyoxyethylene (20) cetostearyl ether, polyoxyethylene (40) hydrogenated castor oil, polyoxyethylene (10) oleyl ether, polyoxyethylene (40) stearate, polysorbate 20, polysorbate 40, polysorbate 60, polysorbate 80 (e.g., Tween™ 80 of ICI), propylene glycol laurate (e.g., Lauroglycol™ of Gattefossé), sodium lauryl sulfate, sorbitan monolaurate, sorbitan monooleate, sorbitan monopalmitate, sorbitan monostearate and tyloxapol, and mixtures thereof.

Solution/suspension compositions of the present invention optionally comprise one or more pharmaceutically acceptable suspending agents. Suspending agents are used to impart increased viscosity and retard sedimentation. Suspending agents are of various classes including cellulose derivatives, clays, natural gums, synthetic gums and miscellaneous agents. Non-limiting examples of suspending agents that can be used in compositions of the present invention include acacia, agar, alginic acid, aluminum monostearate, attapulgite, bentonite, carboxymethylcellulose calcium, carboxymethylcellulose sodium, carrageenan, carbomer, for example carbomer 910, dextrin, ethylmethylcellulose, gelatin, guar gum, HPMC, methylcellulose, ethylcellulose, ethylhydroxyethylcellulose, hydroxyethylcellulose, hydroxypropylcellulose, kaolin, magnesium aluminum silicate, microcrystalline cellulose, microcrystalline cellulose with carboxymethylcellulose sodium, powdered cellulose, silica gel, colloidal silicon dioxide, locust bean gum, pectin, sodium alginate, propylene glycol alginate, tamarind gum, tragacanth, xanthan gum, povidone, veegum, glycyrrhizin, pregelatinized starch, sodium starch glycolate and mixtures thereof.

In certain circumstances, it may be desirable to use flocculating agents in compositions of the invention. Solution/suspension compositions of the invention optionally comprise one or more pharmaceutically acceptable flocculating agents. Flocculating agents enable particles to link together in loose aggregates or flocs and include surfactants, hydrophilic polymers, clays and electrolytes. Non-limiting examples of flocculating agents that may be used in compositions of the present invention include sodium lauryl sulfate, docusate sodium, benzalkonium chloride, cetylpyridinium chloride, polysorbate 80, sorbitan monolaurate, carboxymethylcellulose sodium, xanthan gum, tragacanth, methylcellulose, polyethylene glycol, magnesium aluminum silicate, attapulgite, bentonite, potassium dihydrogen phosphate, aluminum chloride, sodium chloride and mixtures thereof.

An embodiment of the present invention is a concentrated composition, either a concentrated solution or a concentrated solution/suspension, that can be directly imbibed, or diluted with inert diluents and/or other carriers and imbibed; such compositions of the invention, whether diluted or not, are referred to for convenience herein as “imbibable compositions”. Imbibable compositions can be prepared by any suitable method of pharmacy which includes the steps of bringing into association the selective COX-2 inhibitory drug, illustratively celecoxib, and the solvent liquid. Celecoxib compositions of this embodiment preferably contain about 40 mg/ml to about 750 mg/ml, more preferably about 50 mg/ml to about 500 mg/ml, still more preferably about 50 mg/ml to about 350 mg/ml, and most preferably, about 100 mg/ml to about 300 mg/ml, for example about 200 mg/ml, of celecoxib.

In a further embodiment, solutions or solution/suspensions of the invention are provided which are already at a dilution suitable for direct, imbibable administration. In this embodiment, solutions or solution/suspensions of the present invention are added, in a therapeutically effective dosage amount, to about 1 ml to about 20 ml of an inert liquid. Preferably, solutions or solution/suspensions of the present invention are added to about 2 ml to about 15 ml, and more preferably to about 5 ml to about 10 ml, of inert liquid. The term “inert liquid” as used herein refers to pharmaceutically acceptable, preferably palatable liquid carriers. Such carriers are typically aqueous. Examples include water, fruit juices, carbonated beverages, etc.

It has been found that the demands of a rapid-onset formulation are met surprisingly well by a preparation containing a solution or solution/suspension of the present invention encapsulated in a discrete dosage unit. Therefore, another embodiment of the present invention is a concentrated composition, either a solution or solution/suspension, wherein said composition is formulated in a discrete dosage unit or units, for example capsules. Such capsules can have a soft or hard wall composed of any suitable pharmaceutical capsule wall material. Suitably, the wall can comprise gelatin and/or HPMC, optionally with one or more plasticizers. In a particular embodiment the discrete dosage units are soft gelatin capsules.

Preferably, one to about six, more preferably one to about four, and still more preferably one or two of such discrete dosage units per day provides a therapeutically effective dose of a selective COX-2 inhibitory drug.

Compositions of this embodiment are preferably formulated such that each discrete dosage unit contains about 0.3 ml to about 1.5 ml, more preferably about 0.3 ml to about 1 ml, for example about 0.8 ml or about 0.9 ml, of solution or solution/suspension.

Concentrated solutions or solutions/suspensions can be encapsulated by any method known in the art including the plate process or the rotary or reciprocating die process. By the rotary die process, liquid gelatin flowing from an overhead tank is formed into two continuous ribbons by a rotary die machine and brought together by twin rotating dies. Simultaneously, metered fill material is injected between ribbons at the same moment that the dies form pockets of the gelatin ribbons. These pockets of fill-containing gelatin are then sealed by pressure and heat, and the capsules are served from the machine. Soft gelatin capsules may be manufactured in different shapes including round, oval, oblong, and tube-shape, among others. Additionally, by using two different ribbon colors, two-tone capsules can be produced.

### EXAMPLES

### Example 1

Solubility of celecoxib and valdecoxib was determined in each of several different solvent liquids as shown in Table 1, below. To determine solubility, a solid sample consisting of a known amount, typically about 50 mg, of celecoxib or valdecoxib powder was weighed into a test tube. Aliquots of a solvent liquid were then added dropwise in approximately 100 mg increments to the solid sample. The resulting mixture was vortexed and/or sonicated between aliquot additions. Aliquots of solvent liquid were added until the solvent liquid was clear, indicating that the sample was completely dissolved. Ranges in Table 1 indicate that the solubility of celecoxib or valdecoxib is between the values given but has not been more precisely determined. Solubility values preceded by the<symbol denote that, at the particular concentration shown, the mixture was still cloudy, i.e., not all of the drug was fully in dissolved form.

TABLE 1
Solubility of celecoxib and valdecoxib in various solvent liquids

| | Solubility of | Solubility of |
|---|---|---|
| Solvent liquid | celecoxib (mg/g) | valdecoxib (mg/g) |
| propylene glycol | 23-41 | 10-20 |
| ethyl caprylate | 25 | |
| propylene glycol laurate | 18 | 22 |
| Labrasol ™ ¹ | 64 | 34 |
| propylene glycol laurate/ | 58 | 42 |
| Labrasol ™ 1:1 w/w | | |
| Capmul ™ MCM² | 19-21 | 13 |
| Miglyol ™ 812³ | 6-12 | |
| Tagat ™ TO⁴ | 24-40 | 23 |
| Tagat ™ TO/ | 34-52 | 24 |
| Capmul ™ MCM 1:1 w/w | | |
| polyethylene glycol 400 | 304 | 50-85 |
| polyethylene glycol 400/ | 6 | 13 |
| water 2:1 w/w | | |
| polyethylene glycol 400/ | <1 | 1 |
| water 1:1 w/w | | |
| diethylene glycol | 350 | 120 |
| monoethyl ether (DGME) | | |
| DGME/water 2:1 w/w | 42 | 32 |
| DGME/water 1:1 w/w | 3 | 6 |
| Labrasol ™/ | 313-325 | |
| DGME/propylene glycol | | |
| laurate 45:45:10 w/w | | |
| Labrasol ™/ | 288-297 | 130 |
| DGME/propylene glycol | | |
| laurate 40:40:20 w/w | | |
| Labrasol ™/ | 266 | |
| DGME/propylene glycol | | |
| laurate 35:35:30 w/w | | |
| Labrasol ™/DGME 1:1 w/w | 335 | |
| Tagat ™/Capmul ™ | 212 | |
| MCM/DGME 35:35:30 w/w | | |
| Tagat ™/Capmul ™ | 274 | |
| MCM/DGME 58:12:30 w/w | | |
| tetraethylene glycol | 188 | |
| dimethyl ether | | |
| triethylene glycol | 170 | |
| monoethyl ether | | |
| polysorbate 80 | 73 | |
| Arlacel ™ 186⁵ | 13 | |
| Cremophor ™ EL⁶ | 36 | |

¹Labrasol ™ = polyoxyethylene (8) caprylic/capric glycerides
²Capmul ™ MCM = caprylic/capric mono- and diglycerides
³Miglyol ™ 812 = caprylic/capric triglycerides
⁴Tagat ™ TO = polyoxyethylene glyceryl trioleate
⁵Arlace ™ 186 = glyceryl monooleate
⁶Cremophor ™ EL = polyoxyethylene (35) castor oil

The data in Table 1 illustrate advantages of the glycol ether solvent DGME for preparation of orally deliverable solutions by comparison with glycol solvents such as propylene glycol and polyethylene glycol, that are known in prior art for preparing parenteral solutions of selective COX-2 inhibitory drugs. For example, solubility of celecoxib in DGME has been determined to be about 304 mg/g, by contrast with solubility of the same drug in propylene glycol, which is only about 23-41 mg/g. A similar approximately tenfold advantage in solubility is shown for DGME over propylene glycol in the case of valdecoxib.

Although the solubility advantage of DGME over polyethylene glycol 400 (PEG-400) as a solvent for celecoxib is less pronounced, a major advantage is seen for DGME when water is added to the solvent liquid. Solubility of celecoxib in a DGME/water mixture is significantly higher than in a PEG-400/water mixture at the same ratio of mixture ingredients. Without being bound by theory, it is believed that in the aqueous environment of the gastrointestinal tract, significantly more celecoxib will remain in solution, and hence available for immediate absorption, when delivered in a DGME-based solvent liquid than when the solvent liquid is based on PEG-400.

### Example 2

Soft gelatin encapsulated formulations F1, F3, F4, F5, F7, F8, F9 and F10 were prepared having components as shown in Table 2, below. Each formulation was hand-filled into soft gelatin capsules in a final amount of 0.9 g or 0.8 g, containing 200 mg of celecoxib, per capsule, and sealed.

TABLE 2
Composition (mg/capsule) of soft gelatin capsule formulations

| Formulation No. | F1 | F3 | F4 | F5 | F7 | F8 | F9 | F10 |
|---|---|---|---|---|---|---|---|---|
| celecoxib | 200 | 200 | 200 | 200 | 200 | 200 | 200 | 200 |
| Labrasol ™¹ | 280 | — | 350 | — | — | — | — | 240 |
| DGME | 280 | 210 | 350 | 210 | 280 | 240 | 180 | 240 |
| Tagat ™ TO² | — | 245 | — | 406 | 350 | 300 | 348 | — |
| Capmul ™ MCM³ | — | 245 | — | 84 | 70 | 60 | 72 | — |
| propylene glycol laurate | 140 | — | — | — | — | — | — | 120 |
| Total | 900 | 900 | 900 | 900 | 900 | 800 | 800 | 800 |

¹Labrasol ™ = polyoxyethylene (8) caprylic/capric glycerides
²Tagat ™ TO = polyoxyethylene glyceryl trioleate
³Capmul ™ MCM = caprylic/capric mono- and diglycerides

### Example 3

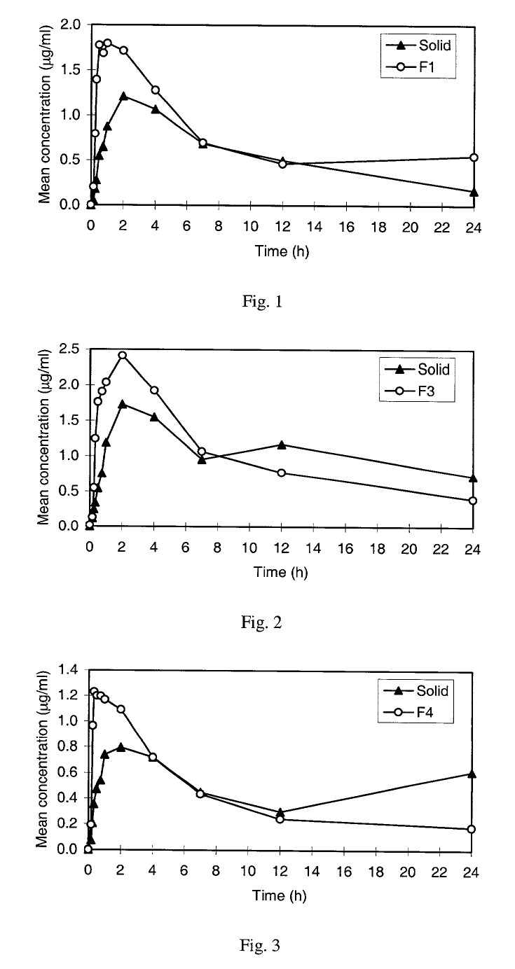

A study was performed in order to determine pharmacokinetic properties of celecoxib formulations F1, F3 and F4 of Example 2, in male beagle dogs. Twenty four dogs (Marshall Farms, North Pose, N.Y.) weighing approximately 7 to 9 kg and approximately 15 to 19 months of age were randomly divided into three groups and acclimated for 5 days. The general environment was maintained as follows: temperature 18.3° C.; humidity 40% or greater; approximately a 12-hour light, 12-hour dark cycle. The dogs were fasted overnight prior to dosing and for at least 4 hours post-dose. PMI Certified Canine Chow Diet # 5007 (PMI Nutrition Inc., Brentwood, Mo.) was available ad libitum to the animals throughout the study. Water from a reverse-osmosis water system was also available ad libitum. Each group received an oral dose of solid celecoxib in capsule form for comparison, followed by an oral dose of formulation F1, F3 or F4, in a two-way cross-over design. A five day washout period was provided between doses. Celecoxib was administered at a dose of 200 mg per animal and venous blood was collected pre-dose, and at 10, 15, 20, 30 and 45 minutes and 1, 2, 4, 7, 12 and 24 hours post-dose. Plasma was separated from blood by centrifugation at 3000×G and samples were stored at −20° C. until analysis. Concentrations of celecoxib in plasma were determined using an HPLC assay. Results are shown in FIGS. 1, 2 and 3.

In general, solvent liquid compositions containing diethylene glycol monoethyl ether and formulated in soft gelatin capsules exhibited superior rapid-onset pharmacokinetic profiles compared to solid capsule formulations. For example, overall, the soft gelatin capsules exhibited higher maximum plasma concentrations (Cₘₐₓ), and faster time to maximum plasma concentration (Tₘₐₓ).

### Example 4

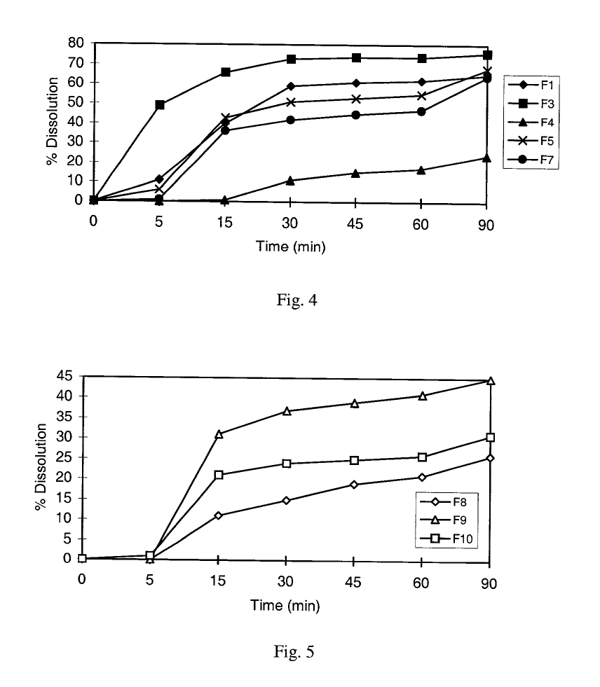

Celecoxib dissolution rates were measured in vitro for each of the soft gelatin capsule formulations described in Example 2, in a standard USP dissolution assay under the following conditions. USP apparatus II paddles were used to stir a dissolution medium (1 liter water containing 1% sodium dodecyl sulfate) at a speed of 75 rpm and a temperature of 37° C. After stirring for 90 minutes, an infinity time point was achieved by stirring at 250 rpm. The medium was then filtered through 10 mm Van-Kel filters. Samples were analyzed for celecoxib via UV detection. Dissolution rates for each of the formulations are shown in FIGS. 4 and 5.

It will be understood that in vitro dissolution rates obtained by the above procedure are not necessarily indicative in absolute terms of the process of release of celecoxib from an encapsulated solution in the gastrointestinal tract. However, it is believed that in relative terms a formulation exhibiting more rapid or complete dissolution in this assay will provide faster release in the gastrointestinal tract, and thereby faster onset of therapeutic effect.

It will be noted in FIG. 4 that among the 900 mg capsule formulations containing 200 mg celecoxib, the most rapid and complete in vitro dissolution was obtained with F3, wherein the solvent liquid comprises DGME accompanied by two co-solvents, polyoxyethylene glyceryl trioleate (Tagat™ TO) and caprylic/capric mono- and diglycerides (Capmul™ MCM).

What is claimed is:

1. An orally deliverable pharmaceutical composition comprising a selective cyclooxygenase-2 inhibitory drug of low water solubility and a solvent liquid that comprises a pharmaceutically acceptable glycol ether, wherein at least a substantial part of the drug is in dissolved or solubilized form in the solvent liquid.

2. The composition of claim 1 wherein the selective cyclooxygenase-2 inhibitory drug is selected from celecoxib, deracoxib, valdecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid and 2-(3,4-difluorophenyl)-4-(3-hydroxy-3-methyl-1-butoxy)-5-[4-(methylsulfonyl)phenyl]-3-(2H)-pyridazinone.

3. The composition of claim 1 wherein the selective cyclooxygenase-2 inhibitory drug is celecoxib.

4. The composition of claim 3 that comprises one or more dosage units each comprising about 10 mg to about 400 mg of celecoxib.

5. The composition of claim 3 having a concentration of celecoxib of about 1% to about 75% by weight.

6. The composition of claim 1 wherein the glycol ether is of formula
R¹—O—((CH₂)ₘO)ₙ—R²
wherein R¹ and R² are independently hydrogen or C₁₋₆ alkyl, C₁₋₆ alkenyl, phenyl or benzyl groups, no more than one of R¹ and R² being hydrogen; m is an integer of 2 to about 5; and n is an integer of 1 to about 20.

7. The composition of claim 6 wherein, in the formula for said glycol ether, one of R¹ and R² is a C₁₋₄ alkyl group and the other is hydrogen or a C₁₋₄ alkyl group.

8. The composition of claim 6 wherein, in the formula for said glycol ether, one of R¹ and R² is a methyl or ethyl group and the other is hydrogen or a methyl or ethyl group.

9. The composition of claim 6 wherein, in the formula for said glycol ether, m is 2.

10. The composition of claim 6 wherein, in the formula for said solvent, n is 1 to about 4.

11. The composition of claim 6 wherein the glycol ether is selected from ethylene glycol monomethyl ether, ethylene glycol dimethyl ether, ethylene glycol monoethyl ether, ethylene glycol diethyl ether, ethylene glycol monobutyl ether, ethylene glycol dibutyl ether, ethylene glycol monophenyl ether, ethylene glycol monobenzyl ether, ethylene glycol butylphenyl ether, ethylene glycol terpinyl ether, diethylene glycol monomethyl ether, diethylene glycol dimethyl ether, diethylene glycol monoethyl ether, diethylene glycol diethyl ether, diethylene glycol divinyl ether, ethylene glycol monobutyl ether, diethylene glycol dibutyl ether, diethylene glycol monoisobutyl ether, triethylene glycol dimethyl ether, triethylene glycol monoethyl ether, triethylene glycol monobutyl ether, tetraethylene glycol dimethyl ether, and mixtures thereof.

12. The composition of claim 6 wherein the glycol ether is diethylene glycol monoethyl ether.

13. The composition of claim 1 wherein substantially all of the selective cyclooxygenase-2 inhibitory drug present in the composition is in dissolved or solubilized form.

14. The composition of claim 13 wherein the solvent liquid comprises (a) a pharmaceutically acceptable glycol ether; and (b) one or more excipients selected from co-solvents, sweeteners, antioxidants, preservatives, buffering agents, flavoring agents, colorants and thickeners.

15. The composition of claim 14 wherein the glycol ether is diethylene glycol monoethyl ether and the solvent liquid further comprises one or more excipients selected from polyoxyethylene (8) caprylic/capric glycerides, caprylic/capric mono- and diglycerides, propylene glycol laurate and polyoxyethylene glyceryl trioleate.

16. The composition of claim 13 wherein the solvent liquid comprises diethylene glycol monoethyl ether, caprylic/capric mono- and diglycerides, and polyoxyethylene glyceryl trioleate.

17. The composition of claim 1 wherein a first substantial portion of the selective cyclooxygenase-2 inhibitory drug present in the composition is in dissolved or solubilized form, and the composition further comprises a second portion of the selective cyclooxygenase-2 inhibitory drug in particulate form dispersed in the solvent liquid.

18. The composition of claim 17 wherein said first portion comprises at least about 15% by weight of the selective cyclooxygenase-2 inhibitory drug present in the composition.

19. The composition of claim 17 wherein the solvent liquid comprises (a) a pharmaceutically acceptable glycol ether; and (b) one or more excipients selected from co-solvents, wetting agents, suspending agents, flocculating agents, sweeteners, antioxidants, preservatives, buffering agents, flavoring agents, colorants and thickeners.

20. The composition of claim 17 wherein said second portion of the selective cyclooxygenase-2 inhibitory drug has a D₉₀ particle size less than about 200 μm.

21. The composition of claim 1 that is an unencapsulated imbibable liquid.

22. The composition of claim 1 that comprises one or more discrete dosage units, wherein a therapeutically effective amount of the selective cyclooxygenase-2 inhibitory drug is contained in one to a small plurality of said dosage units.

23. The composition of claim 22 wherein the dosage units are liquid-filled capsules having a wall.

24. The composition of claim 23 wherein the wall comprises gelatin and/or hydroxypropylmethylcellulose.

25. The composition of claim 22 wherein the dosage units are soft gelatin capsules.

26. The composition of claim 25 wherein each capsule contains about 0.3 ml to about 1.5 ml of the composition.

27. The composition of claim 1 that further comprises a vasomodulator, wherein the selective cyclooxygenase-2 inhibitory drug and the vasomodulator are present in total and relative amounts effective to relieve pain in headache or migraine.

28. The composition of claim 1 that further comprises an alkylxanthine compound, wherein the selective cyclooxygenase-2 inhibitory drug and the alkylxanthine compound are present in total and relative amounts effective to relieve pain in headache or migraine.

29. The composition of claim 28 wherein the alkylxanthine compound is selected from caffeine, theophylline and theobromine.

30. The composition of claim 28 wherein the alkylxanthine compound is caffeine.

31. A method of treating a medical condition or disorder in a subject where treatment with a cyclooxygenase-2 inhibitor is indicated, comprising orally administering to the subject a composition of claim 1.

32. A method of analgesia comprising orally administering, to a subject in need of analgesia, an effective pain-relieving amount of a composition of claim 1 comprising a selective cyclooxygenase-2 inhibitory drug.

33. The method of claim 32 wherein the subject suffers from headache or migraine and wherein there is further orally administered to the subject a vasomodulator, the selective cyclooxygenase-2 inhibitory drug and the vasomodulator being administered in total and relative amounts effective to relieve pain in the headache or migraine.

34. The method of claim 33 wherein the vasomodulator is coformulated with the selective cyclooxygenase-2 inhibitory drug.

35. The method of claim 32 wherein the subject suffers from headache or migraine and wherein there is further orally administered to the subject an alkylxanthine compound, the selective cyclooxygenase-2 inhibitory drug and the alkylxanthine compound being administered in total and relative amounts effective to relieve pain in the headache or migraine.

36. The method of claim 35 wherein the alkylxanthine compound is coformulated with the selective cyclooxygenase-2 inhibitory drug.

37. The method of claim 35 wherein the alkylxanthine compound is selected from caffeine, theophylline and theobromine.

38. The method of claim 35 wherein the alkylxanthine compound is caffeine.
